(19) United States
(12) Patent Application Publication
(10) Pub. No.: US 2007/0154923 A1
(43) Pub. Date: Jul. 5, 2007

(54) Method for Gridding and Quality Control of Polymer Arrays

(75) Inventors: Paul C. Ciccolella, San Jose, CA (US); Ryan Estrada, Fairfield, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, Inc., Santa Clara, CA (US)

(21) Appl. No.: 11/617,534

(22) Filed: Dec. 28, 2006

Related U.S. Application Data
(60) Provisional application No. 60/754,693, filed on Dec. 29, 2005.

(51) Int. Cl. C12Q 1/68

(52) U.S. Cl. 435/6; 977/924

(57) ABSTRACT

Methods of using a plurality of control probes for gridding of a nucleic acid array and for quality control purposes are disclosed.

### FIELD OF THE INVENTION

The present invention relates to methods of making microarrays having known probe sequences at predefined locations on a microarray substrate where the probe sequences can be scanned for purposes of gridding the array and/or quality control. The present invention also relates to the microarrays themselves having probe sequences at predefined regions on the array that are used for gridding or quality control.

### BACKGROUND OF THE INVENTION

A variety of systems are known for synthesizing or depositing dense arrays of biological materials, sometimes referred to as probes, on a substrate or support. Labeled targets in hybridized probe-target pairs may be detected using various commercial devices, referred to for convenience hereafter as scanners. Scanners image the targets by detecting fluorescent or other emissions from the labels. Data representing the detected emissions are stored in a memory device for processing. The processed images may be presented to a user on a video monitor or other device, and/or operated upon by various data processing products or systems. Some techniques are known for identifying the data representing detected emissions and separating them from background information. For example, U.S. Pat. No. 6,090,555 to Fiekowsky, et al., hereby incorporated by reference in its entirety for all purposes, describes various techniques. Also, the use of certain probe sequences for either gridding or quality control purposes is also known. See U.S. Pat. No. 6,927,032 hereby incorporated by reference in its entirety. See also US 2004/0175719 A1 hereby incorporated by reference in its entirety for all purposes which describes synthetic Tag gene sequences for use in assay development, in product development and validation, and for quality control. See also Manufacturing Quality Control and Validation Studies of GeneChip Arrays. Affymetrix Technical Note 2002 hereby incorporated by reference in its entirety for all purposes.

Devices and computer systems for forming and using arrays of materials on a chip or substrate are known. For example, PCT applications WO92/10588 and 95/11995, both incorporated herein by reference for all purposes, describe techniques for sequencing or sequence checking nucleic acids and other materials. Arrays for performing these operations may be formed according to the methods of, for example, the pioneering techniques disclosed in U.S. Pat. Nos. 5,445,934, 5,384,261 and 5,571,639, each incorporated herein by reference for all purposes.

According to one aspect of the techniques described therein, an array of nucleic acid probes is fabricated at known locations on a chip. A labeled nucleic acid is then brought into contact with the chip and a scanner generates an image file indicating the locations where the labeled nucleic acids are bound to the chip. Based upon the image file and identities of the probes at specific locations, it becomes possible to extract information such as the nucleotide or monomer sequence of DNA or RNA. Such systems have been used to form, for example, arrays of DNA that may be used to study and detect mutations relevant to genetic diseases, cancers, infectious diseases, HIV, and other genetic characteristics.

The VLSIPS™ technology provides methods of making very large arrays of oligonucleotide probes on very small chips. See U.S. Pat. No. 5,143,854 and PCT patent publication Nos. WO 90/15070 and 92/10092, each of which is incorporated by reference for all purposes. The oligonucleotide probes on the DNA probe array are used to detect complementary nucleic acid sequences in a sample nucleic acid of interest (the “target” nucleic acid).

For sequence checking applications, the chip may be tiled for a specific target nucleic acid sequence. As a example, the chip may contain probes that are perfectly complementary to the target sequence and probes that differ from the target sequence by a single base mismatch. For de novo sequencing applications, the chip may include all the possible probes of a specific length. The probes are tiled on a chip in rows and columns of cells, where each cell includes multiple copies of a particular probe. Additionally, “blank” cells may be present on the chip which do not include any probes. As the blank cells contain no probes, labeled targets should not bind specifically to the chip in this area. Thus, a blank cell provides a measure of the background intensity.

Although the photolithographic equipment for synthesizing chips is extremely accurate, occasionally variations occur in the manufacturing process. For example, errors may occur if a chemical is not properly added, a wash step is skipped, concentrations are not correct, timing is incorrect, the wrong mask is utilized, the correct mask is misaligned, and the like. It is often very difficult to detect any errors at all and many of the errors only affect a small limited number of probes on the chip. For stringent quality control, for example, it would be desirable to detect variations in the manufacturing process before the chips are shipped to customers. Additionally, it would be desirable to have an indication of wnat was the cause of the error so that it can be corrected. Techniques addressing these issues are known. See for example US 2005/0216201 hereby incorporated by reference in its entirety for all purposes

In the scanned image file, a cell is typically represented by multiple pixels. Although a visual inspection of the scanned image file may be performed to identify the individual cells in the scanned image file, it would be desirable to utilize computer-implemented image processing techniques to align the scanned image. Such computer image processing steps are known. See for example U.S. Pat. No. 6,090,555 hereby incorporated by reference in its entirety for all purposes.

Gridding is used to identify the location of probes in a scanned image. A grid is aligned over the scanned image and a particular pattern. Pixels that correspond to each cell can then be identified. The pattern in the scanned image can be a checkerboard pattern that is generated by synthesizing alternating cells that include probes that are complementary to a control nucleic acid sequence. The control nucleic acid sequence may be a known sequence that is labeled and hybridized to the chip for the purpose of aligning the scanned image. Additionally, the brightness of the cells complementary to the control nucleic acid sequence may be utilized as a baseline or for comparison to other intensities.

Methods of automated gridding are known. See for example Bentley et al., “The Development and Application of Automated Gridding for Efficient Screening of Yeast and Bacterial Ordered Libraries,” Genomics 12, 1992, pp. 534-541. Other methods of gridding are known. See for example U.S. Pat. No. 6,829,376 hereby incorporated by reference in its entirety for all purposes which describes methods of aligning a grid with a checkerboard image.

### SUMMARY OF THE INVENTION

Embodiments of the present invention are directed to the manufacture of known probe sequences on preselected regions on a microarray substrate. The known probe sequences are used for gridding or quality control purposes. Known target molecules, including a detectable moiety, are hybridized to the known probe sequences. The array is then scanned to detect the detectable moieties and accordingly, the extent of hybridization.

The extent of hybridization of the target molecule to the known probe sequences can be correlated with the quality of the microarray as a whole or can further be used to align a grid onto the microarray surface for the purpose of identifying probes at certain locations on the array. The use of known probe sequences provides useful information on design verification, synthesis verification, gridding and signal intensity and in certain circumstances without having to scan the entire array.

According to one embodiment of the present invention, an array is designed such that probes of known identity are to be placed at defined x,y locations on an array substrate. The probes are then synthesized on the array using methods, such as the photolithographic and combinatorial chemistry methods described in U.S. Pat. Nos. 5,571,639, 5,593,839 and 5,856,101. According to one aspect, a substrate, such as a quartz wafer, includes linker molecules bound to the surface of the substrate. The linker molecules each have one or more light-removable protecting groups. Removal of the protecting group (referred to as “deprotection”) activates the linker molecule for chemical coupling with a subsequently added monomer compound which also has one or more light removable protecting groups. The substrate is coated with a light sensitive compound that inhibits coupling between the substrate and a monomer of the probe being constructed. Lithographic masks are used to block or transmit light onto specific locations of the substrate surface. Light-removable protecting groups are removed at locations where light contacts the substrate surface to create activated regions, i.e. regions capable of reacting with a subsequently added monomer due to removal of the light removable protecting group. The substrate surface is then contacted with a monomer and coupling occurs at the activated regions on the substrate surface. Since the monomer includes a light-removable protecting group, according to one embodiment of the invention, the cycle of deprotection and monomer addition can be repeated any number of times to create desired probes at desired locations and densities on a particular array substrate. According to alternate embodiments, preformed probes can be bound to the surface of the array, as opposed to being synthesized by a monomer-by-monomer method. Furthermore, arrays can be manufactured by different methods including directly spotting preformed probes, oligomers or monomers to the substrate surface. Methods of making arrays included within the scope of the present invention are discussed below.

In order to determine the quality of array manufacture or to facilitate gridding of the array from a scanned image, quality control probes are synthesized on the array at designated locations in conjunction with the predetermined array probes. The quality control probes are hybridized with labeled compounds, such as fluorescent compounds, and then scanned. The degree of hybridization measured by the fluorescence is a measure of the quality of the array and the extent of hybridization of the array as a whole. The image can also be used to grid or align the array so that the location of probes can be determined.

According to one aspect of the present invention, a method of monitoring hybridization of control sample sequences to control probe sequences on an array is described. According to the method, a high density polymer probe array is provided that has a plurality of test probes that bind to test sample targets and a plurality of control probes at predefined regions on the array and wherein the control probes bind to control sample targets. The control probes further include a plurality of first control probes at predefined regions on the array and a plurality of second control probes at predefined regions on the array. According to one aspect, the first control probes are different from the second control probes, and accordingly, a given sample target will not bind to both the first and second control probes. According to the method, first control sample targets are contacted with the array. Whether hybridization occurs between the first control sample targets and the first control probes is determined. By using certain control sample targets, hybridization to certain control probes can be selective to the exclusion of other control probes. This can be important, for example, in gridding applications where hybridization to other than the selected control probe region can adversely effect grid placement, and accordingly, probe identification.

According to one embodiment, the first control probes are unique to the array when compared to other probes and second control probes present on the array surface. In one embodiment, the first control probes can be of any sequence so long as they are different from the remaining probes on the array substrate. Since the first control probes are different from other sequences on the array, hybridization can be limited to the first control probes.

According to an additional embodiment of the invention, a method for gridding or quality control of an oligonucleotide array is provided. The array has a plurality of quality control blocks surrounded at the four corners of each quality control block with first checkerboard features. The terms “blocks” and “features” refers to the probe sequences on the array at defined regions. According to an aspect of the method, a quality control block is provided at the center of the array and second checkerboard features are provided samples that are capable of hybridizing or binding to the first checkerboard features do not hybridize or bind to the second checkerboard features and vice versa. The center control blocks and the second checkerboard features are imaged to allow for gridding of the array and determining whether the probes in the quality control blocks have been accurately synthesized. The use of distinct checkerboard features for gridding of the array advantageously allows for an increased scan area of the array for gridding purposes without having other checkerboard patterns not associate with gridding interfering with the gridding algorithm used by the software.

### DETAILED DESCRIPTION OF THE INVENTION

Embodiments of the present invention are directed to the use of two or more sets of control probes located at predefined regions of the array in selected patterns. The methods of the present invention are useful in gridding or aligning the array and in determining the extent of hybridization to the array given variations in array manufacture.

According to one embodiment of the present invention, control probes are located on the array at predefined regions and can be advantageously scanned without having to scan the entire array which reduces time spent on quality control analysis. A further embodiment of the invention includes separate sets of control probes which are different from each other, such that hybridization to one set of probes provides different quality control information from hybridization to a different set of control probes. According to one aspect, different sets of control probes are located at different predefined regions on the array. In this manner, different regions of the array can be scanned to measure different quality control factors and to also allow gridding of the array without interference from other control probes that may be in the same or similar predefined region of the array.

According to one aspect of the present invention, a method of monitoring hybridization of control sample sequences to control probe sequences on an array is described. According to the method, a high density polymer probe array is provided that has a plurality of test probes that bind to test sample targets and a plurality of control probes at predefined regions on the array and wherein the control probes bind to control sample targets. The control probes further include a plurality of first control probes at predefined regions on the array and a plurality of second control probes at predefined regions on the array. According to one aspect, the first control probes are different from the second control probes. According to the method, first control sample targets are contacted with the array. Whether hybridization occurs between the first control sample targets and the first control probes is determined.

First control probes of the present invention can include any sequences that are unique to a particular predefined region on an array. A particular binding sequence, preferably including a detectable marker, is used to hybridize with the first control probes. Accordingly, when scanned, only that predefined region having the first control probes should be the source of detectable marker should hybridization have occurred. This region can then be advantageously used to grid the array without interference from other control probes that may also be in the same or similar area on the array.

According to one embodiment of the present invention, first control probes can be any sequence that is unique to the array, i.e. that is not found at any other area of the array other than the predefined region for the first control probes or at least is not found at an area of the array which would interfere or overlap with detected emissions from the predefined region for the first control probes. Such probes can include synthetic sequences, and preferably synthetic gene sequences such as synthetic Tag gene sequences. Such synthetic Tag sequences within the scope of the present invention include those described in US 2004/0175719 hereby incorporated by reference herein for all purposes. Other probes can include probe sequences that bind to gene sequences such as bacterial gene sequences including Bio B, C, D and cre and those described in U.S. Pat. No. 6,927,032 hereby incorporated by reference in its entirety for all purposes.

According to certain aspects of the present invention, second control probes are advantageously employed in the methods described herein. Second control probes are those which include probe sequences that bind to gene sequences such as bacterial gene sequences including Bio B, C, D and cre and those described in U.S. Pat. No. 6,927,032 hereby incorporated by reference in its entirety for all purposes. Other probes can include synthetic sequences, and preferably synthetic gene sequences such as synthetic Tag gene sequences. Such synthetic Tag sequences within the scope of the present invention include those described in US 2004/0175719 hereby incorporated by reference herein for all purposes. According to one aspect of the invention, the first and second control probes are different. According to another aspect, the first control probes are unique to a subset of checkerboard features.

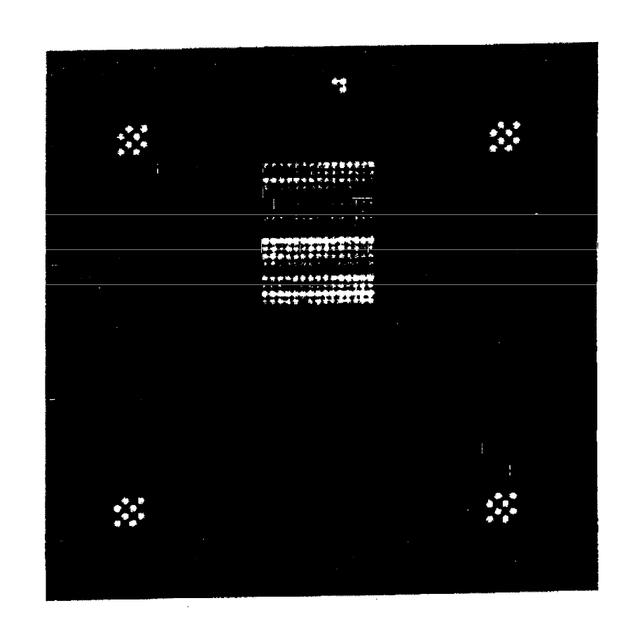

Accordingly to certain aspect of the present invention, the first control probes and the second control probes may be arranged at predefined regions of the array. One such exemplary arrangement is shown in FIG. 1. FIG. 1 shows a block feature located at a center location on the array and having four checkerboard pattern feature locations at or near the four corners of the center block. Block features may be located on the array without checkerboard features and checkerboard features may be present on the array without block features. Checkerboard features and block features may be closely spaced on the array to one another and to each other. According to one embodiment of the present invention, first control probes are present at the four checkerboard locations while second control probes are present at the center block location. This arrangement advantageously allows separate quality control measures to be directed to the center block versus the four checkerboard locations. However, the checkerboard features and the center block features can include the same probe sequence. According to an additional aspect of the present invention, additional checkerboard features may be placed at or near the center block with the additional checkerboard features having probes different from the first control probes and center block probes. In this manner, one can direct each checkerboard or a subset of checkerboards to a separate aspect of quality control or gridding without interference from emissions from other checkerboards or other control sequences.

According to an alternate embodiment of the invention, a method for quality control of an oligonucleotide array is provided whereby the array has a plurality of quality control block features surrounded at the four corners of each quality control block with first checkerboard features. According to an aspect of the method, a quality control block feature is provided at the center of the array and second checkerboard features are provided at the four corners of the center control block. The second checkerboard features can be located near the first checkerboard features. According to a still further aspect, labeled nucleic acid sequences that hybridize to the first checkerboard features do not hybridize to the second checkerboard features. Stated differently, nucleic acids that hybridize to the second checkerboard features preferably do not hybridize to the first checkerboard features. The center control blocks and the second checkerboard features are imaged to allow for gridding of the array and determining whether the probes in the quality control blocks have been accurately synthesized. According to an aspect of the invention, the second checkerboard features will be detected while the first checkerboard features will not be detected. A grid can then be placed over the second checkerboard features and gridding of the array can then take place without interference from other checkerboard features. This aspect of the invention is particularly advantageous when gridding highly dense arrays where checkerboard features can be near to one another and accordingly, detected emissions can overlap and adversely effect the gridding process.

As noted, commercial systems are available for synthesizing or depositing dense arrays of biological materials on a substrate or support. For example, Affymetrix® GeneChip® arrays, manufactured by Affymetrix, Inc. of Santa Clara, Calif., are synthesized in accordance with various techniques sometimes referred to as VLSIPS™ (Very Large Scale Immobilized Polymer Synthesis) technologies. Some aspects of VLSIPS™ technologies are described in U.S. Pat. No. 5,143,854 to Pirrung, et al.; U.S. Pat. No. 5,445,934 to Fodor, et al.; U.S. Pat. No. 5,744,305 to Fodor, et al.; U.S. Pat. No. 5,831,070 to Pease, et al.; U.S. Pat. No. 5,837,832 to Chee, et al.; U.S. Pat. No. 6,022,963 to McGall, et al.; U.S. Pat. No. 6,083,697 to Beecher, et al. Each of these patents is hereby incorporated by reference in its entirety. Generally, the probes of these arrays consist of oligonucleotides synthesized by methods that include the steps of activating regions of a substrate and then contacting the substrate with a selected monomer solution. The regions are activated with a light source shown through a mask in a manner similar to photolithography techniques used in the fabrication of integrated circuits. Other regions of the substrate remain inactive because the mask blocks them from illumination. By repeatedly activating different sets of regions and contacting different monomer solutions with the substrate, a diverse array of polymers is produced on the substrate. Various other steps, such as washing unreacted monomer solution from the substrate, are employed in various implementations of these methods. For convenience, arrays synthesized according to these techniques, or ones now existing or that may be developed in the future based on a synthesis process, are referred to hereafter as synthesized arrays.

Other techniques exist for depositing probes on a substrate or support. For example, spotted arrays are commercially fabricated on microscope slides. These arrays consist of liquid spots containing biological material of potentially varying compositions and concentrations. For instance, a spot in the array may include a few strands of short oligonucleotides in a water solution, or it may include a high concentration of long strands of complex proteins. The Affymetrix® 417™, 427™, and 437™ Arlayers from Affymetrix, Inc. are devices that deposit densely packed spotted arrays of biological material on a microscope slide in accordance with these techniques, aspects of which are described in PCT Application No. PCT/US99/00730 (published on Jul. 22, 1999 as International Publication Number WO 99/36760), hereby incorporated by reference in its entirety. Other techniques for generating spotted arrays also exist. For example, U.S. Pat. No. 6,040,193 to Winkler, et al. is directed to processes for dispensing drops to generate spotted arrays. The '193 patent, and U.S. Pat. No. 5,885,837 to Winkler, also describe the use of micro-channels or micro-grooves on a substrate, or on a block placed on a substrate, to synthesize arrays of biological materials. These patents further describe separating reactive regions of a substrate from each other by inert regions and spotting on the reactive regions. The '193 and '837 patents are hereby incorporated by reference in their entireties. Another technique is based on ejecting jets of biological material to form a spotted array. Various implementations of the jetting technique may use devices such as syringes or piezo electric pumps to propel the biological material.

Spotted arrays or synthesized arrays typically are used in conjunction with tagged biological samples such as cells, proteins, genes, DNA sequences, or other biological elements. These samples, referred to herein as targets, are processed so that they are spatially associated with certain probes in the probe array. For example, one or more chemically tagged biological samples, i.e., the targets, are distributed over the probe array. Some targets hybridize with at least partially complementary probes and remain at the probe locations, while non-hybridized targets are washed away. These hybridized targets, with their tags or labels, are thus spatially associated with the targets' complementary probes. The hybridized probe and target may sometimes be referred to as a probe-target pair. Detection of these pairs can serve a variety of purposes. For example, detection of these pairs on appropriately synthesized arrays can be used to determine whether a target nucleic acid has a nucleotide sequence identical to or different from a specific reference sequence. See, for example, U.S. Pat. No. 5,837,832 to Chee, et al., incorporated by reference herein in its entirety for all purposes. Other uses include gene expression monitoring (see U.S. Pat. Nos. 5,800,992 and 6,040,138, hereby incorporated by reference herein in their entireties), genotyping (see U.S. Pat. No. 5,856,092, hereby incorporated by reference herein in its entirety), or other detection of nucleic acids.

To ensure proper interpretation of the term probe as used herein, it is noted that contradictory conventions exist in the relevant literature. The word probe is used in some contexts to refer not to the biological material that is synthesized on a substrate or deposited on a slide, as described above, but to what has been referred to herein as the target. To avoid confusion, the term probe is used consistently herein to refer to the polymers, such as oligonucleotides, synthesized according to the VLSIPS™ technology, the biological materials deposited so as to create spotted arrays, and materials synthesized or deposited to form arrays according to similar technologies that now exist or may be developed in the future. Thus, arrays formed in accordance with any of these technologies may be referred to generally and collectively hereafter for convenience as probe arrays.

Labeled targets in hybridized probe-target pairs may be detected using any of a variety of commercial scanners. Scanners image the targets by detecting fluorescent or other emissions from the labels, or by detecting transmitted, reflected, or scattered radiation. These processes are generally and collectively referred to hereafter for convenience simply as involving the detection of emissions. FIG. 1 depicts emissions that have been detected by a scanner. The regions are shown to be a center block of emissions with four checkerboard emissions located near the four corners of the center block. An additional region of emissions is shown above the center block. The emissions can result from each region having the same probe and being hybridized by the same labeled test sample. Alternatively, the checkerboards can have probes different from the center block and the emissions can result from different labeled test samples being applied to the array. Various detection schemes are employed depending on the type of emissions and other factors. A typical scheme employs optical and other elements to provide excitation light and to selectively collect the emissions. Also generally included are various light-detector systems employing photodiodes, charge-coupled devices, photomultiplier tubes, or similar devices to register the collected emissions. For example, a scanning system for use with a fluorescent label is described in U.S. Pat. No. 5,143,854, incorporated by reference above. Other scanners or scanning systems are described in U.S. Pat. Nos. 5,578,832; 5,631,734; 5,834,758; 5,981,956 and 6,025,601, and in PCT Application PCT/US99/06097 (published as WO99/47964), each of which is hereby incorporated herein by reference in its entirety for all purposes.

The scanning system provides data representing the intensities (and possibly other characteristics, such as color) of the detected emissions, as well as the locations on the substrate where the emissions were detected. The data typically are stored in a memory device in the form of a data file. One type of data file, sometimes referred to as an image file, typically includes intensity and location information corresponding to elemental sub-areas of the scanned substrate. The term elemental in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the representing another characteristic, such as color. For instance, a scanned elemental sub-area in which high-intensity emissions were detected may be represented by a pixel having high luminance (hereafter, a bright pixel), and low-intensity emissions may be represented by a pixel of low luminance (a dim pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known.

Probes need not have been synthesized or deposited on all areas of the substrate. On an Affymetrix® GeneChip® array, for example, the synthesized region of the entire nucleic acid array typically is bordered by a non-synthesized area. Because the non-synthesized area does not contain probes, labeled targets do not hybridize there and the scanning system ideally should not detect emissions. however, various imperfections may give rise to the detection of background emissions from non-synthesized areas, or from synthesized areas in which hybridization has not occurred. The term background noise may be used hereafter to refer to these detected background emissions and noise from other sources.

Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided to a computer that associates the locations where hybridized targets were detected with known locations where probes of known identities were synthesized or deposited. Information such as the nucleotide or monmer sequence of target DNA or RNA may then be deduced. Techniques for making these deductions are described, for example, in U.S. Pat. No. 5,733,729 to Lipshutz, which hereby is incorporated by reference in its entirety for all purposes, and in U.S. Pat. No. 5,837,832, noted and incorporated above. Among other purposes, the data may be used to study genetic characteristics and to detect mutations relevant to genetic and other diseases or conditions.

In order to facilitate computer processing of the pixel information, it therefore typically is desirable to automate the identification of pixels having particular characteristics and relate them to the known locations of probes. To this end, computer-implemented image processing techniques have been developed to align the scanned image file. That is, the techniques attempt to identify which pixels represent emissions from which probes, and to distinguish this information from background noise. Some of these techniques are described in U.S. Pat. No. 6,090,555 to Fiekowsky, et al., incorporated by reference above. Generally speaking, one aspect of the technique described in the '555 patent operates on a pattern, such as a checkerboard pattern, that is known to be included in the scanned image. When the image is convolved with a filter, a recognizable pattern, such as a grid pattern, is generated in the convolved image. The scanned image may then be aligned according to the position of the recognizable pattern in the convolved image. In some implementations of these techniques, a grid is aligned over the scanned image and the position of the grid is adjusted to minimize a sum of the intensities of pixels along a grid direction.

Applications of this and other techniques may be complicated, however, when extreme, aberrant, or unexpected conditions are encountered during the scanning process, or when features of alignment patterns are close to one another on a given surface area of the array. Accordingly, when the scanner reads several alignment patterns, such as a plurality of checkerboard patterns in the scanned area, the feature of the patterns may overlap. Methods addressing these complications, including difficulties in establishing boundaries between features when they are synthesized or deposited, edge effects that can occur when using photolithographic techniques or from disturbances during the manufacturing process, are discussed in detail in U.S. Pat. No. 6,829,376 hereby incorporated by reference in its entirety for all purposes.

These overlap effects, whatever their cause, may interfere with the implementation of conventional techniques that, for example, search for the boundaries between bright and dim elements in an alignment pattern, or that search between different checkerboard patterns. The unintended result may be that an alignment grid is inaccurately positioned over an image because the grid was itself inaccurately aligned with the alignment pattern.

According to a still further aspect of the invention, an array used in the present invention includes a plurality of regions on the array in which diverse polymer probes are coupled. The array further includes a plurality of control regions having polymer probes coupled thereto where a particular control region has a plurality of the same sequence located therein. The control regions can have any pattern including blocks, squares, checkerboard patterns and combinations thereof. According to one aspect, the control regions are located at the center of the array and can advantageously include a center block and checkerboard patterns located at or near the four corners of the center block.

The present invention has many preferred embodiments and relies on many patents, applications and other references for details known to those of skill in the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent”includes a plurality of agents, including mixtures thereof.

An individual is not limited to a human being, but may also be other organisms including, but not limited to, mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

For purposes of illustration, one embodiment of the present invention takes advantages of making use of a computer system that designs a chip mask, synthesizes the probes on the chip, labels the nucleic acids, and scans the hybridized nucleic acid probes. Such a system is fully described in U.S. Pat. No. 5,571,639 hereby incorporated by reference in its entirety for all purposes.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the specification where indicated. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer. A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been discussed above and are described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of tne above patents, but the same techniques are applied to polypeptide arrays. p Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols. A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675, each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989) Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic acid sample are described in Dong et al., Genome Research 11, 1418 (2001), in U.S. Pat. No. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625 in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758, 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, etc. The computer-executable instructions may be written in a suitable computer language or combination of several languages. Basic computational biology methods are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), Ser. Nos. 10/065,856, 10/065,868, 10/328,818, 10/328,872, 10/423,403, and 60/482,389.

The following definitions are used, unless otherwise described. Halo is fluoro, chloro, bromo, or iodo. Alkyl, alkoxy, aralkyl, alkylaryl, etc. denote both straight and branched alkyl groups; but reference to an individual radical such as “propyl” embraces only the straight chain radical, a branched chain isomer such as “isopropyl” being specifically referred to. Aryl includes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

“Alkyl” refers to a straight chain, branched or cyclic chemical groups containing only carbon and hydrogen. Alkyl groups include, without limitation, ethyl, propyl, butyl, pentyl, cyclopentyl and 2-methylbutyl. Alkyl groups are unsubstituted or substituted with 1 or more substituents (e.g., halogen, alkoxy, amino).

“Alkylene” refers to a straight chain, branched or cyclic chemical group containing only carbon and hydrogen. Alkyl groups include, without limitation, ethylene, propylene, butylene, pentylene, and 2-methylbutylene. Alkyl groups are unsubstituted or substituted with 1 or more substituents (e.g., halogen, alkoxy, amino).

“Aryl” refers to a monovalent, unsaturated aromatic carbocyclic group. Aryl groups include, without limitation, phenyl, naphthyl, anthryl and biphenyl. Aryl groups are unsubstituted or substituted with 1 or more substituents (e.g. halogen, alkoxy, amino). “Arylene” refers to a divalent aryl group.

“Amido” refers to a chemical group having the structure —C(O)NR₃—, wherein R₃ is hydrogen, alkyl or aryl. Preferably, the amido group is of the structure —C(O)NR₃— where R3 is hydrogen or alkyl having from about 1 to about 6 carbon atoms. More preferably, the amido alkyl group is of the structure —C(O)NH—.

“Alkanoyl” refers to a chemical group having the structure —(CH₂)ₙC(O)—, n is an integer ranging from 0 to about 10. Preferably, the alkanoyl group is of the structure —(CH₂)ₙC(O)—, wherein n is an integer ranging from about 2 to about 10. More preferably, the alkanoyl group is of the structur —(CHG₂)ₙC(O)—, wherein n is an integer ranging from about 2 to about 6. Most preferably, the alkanoyl group is of the structure —CH₂C(O)—.

“Alkyl amido” refers to a chemical group having the structure —R4C(O)NR₃—, wherein R₃ is hydrogen, alkyl or aryl, and R₄ is alkylene or arylene. Preferably, the alkyl amido group is of the structure —(CH₂)ₙC(O)NH—, wherein n is an integer ranging from about 1 to about 10. More preferably, n is an integer ranging from about 1 to about 6. Most preferably, the alkyl amido group has the structure —(CH₂)₂C(O)NH— or the structure —CH₂C(O)NH—.

“N-Amido alkyl” refers to a chemical group having the structure —C(O)NR₃R₄—, wherein R₃ is hydrogen, alkyl or aryl, and R₄ is alkylene or arylene. Preferably, the N-amido alkyl group is of the structure —C(O)NH(CH₂)ₙR₅—, wherein n is an integer ranging from about 2 to about 10, and R₅ is O, NR₆, or C(O), and wherein R₆ is hydrogen, alkyl or aryl. More nreferably, the N-amido alkyl group is of the structure —C(O)NH(CH₂)ₙN(H)—, wherein n is an integer ranging from about 2 to about 6. Most preferably, the N-amido alkyl group is of the structure —C(O)NH(CH₂)₄N(H)—.

“Alkynyl alkyl” refers to a chemical group having the structure —C≡C-R₄—, wherein R₄ is alkyl or aryl. Preferably, the alkynyl alkyl group is of the structure —C≡C—(CH₂)ₙR₅—, wherein n is an integer ranging from 1 to about 10, and R₅ is O, NR₆ or C(O), wherein R₆ is hydrogen, alkyl or aryl. More preferably, the alkynyl alkyl group is of the structure —C≡C—(CH₂)ₙN(H)—, wherein n is an integer ranging from 1 to about 4. Most preferably, the alkynyl alkyl group is of the structure —C≡C—CH₂N(H)—.

“Alkenyl alkyl” refers to a chemical group having the structure —CH═CH—R₄—, wherein R₄ is a bond, alkyl or aryl. Preferably, the alkenyl alkyl group is of the structure —CH═CH—(CH₂)ₙR₅—, wherein n is an integer ranging from 0 to about 10, and R₅ is O, NR₆, C(O) or C(O)NR₆, wherein R₆ is hydrogen, alkyl or aryl. More preferably, the alkenyl alkyl group is of the structure —CH═CH—(CH₂)ₙC(O)NR₆—, wherein n is an integer ranging from 0 to about 4. Most preferably, the alkenyl alkyl group is of the structure —CH═CH—C(O)N(H)—.

“Functionalized alkyl” refers to a chemical group of the structure —(CH₂)ₙR₇—, wherein n is an integer ranging from 1 to about 10, and R₇ is O, S, NH or C(O). Preferably, the functionalized alkyl group is of the structure —(CH₂)ₙC(O)—, wherein n is an integer ranging from 1 to about 4. More preferably, the functionalized alkyl group is of the structure —CH₂C(O)—.

“Alkoxy” refers to a chemical group of the structure —O(CH₂)ₙR₈—, wherein n is an integer ranging from 2 to about 10, and R₈ is a bond, O, S, NH or C(O). Preferably, the alkoxy group is of the structure —O(CH₂)ₙ, wherein n is an integer ranging from 2 to about 4. More preferably, the alkoxy group is of the structure —OCH₂CH₂—.

“Alkyl thio” refers to a chemical group of the structure —S(CH₂)ₙR₈—, wherein n is an integer ranging from 1 to about 10, and R₈ is a bond, O, S, NH or C(O). Preferably, the alkyl thio group is of the structure —S(CH₂)ₙ—, wherein n is an integer ranging from 2 to about 4. More preferably, the thio group is of the structure —SCH₂CH₂C(O)—.

“Amino alkyl” refers to a chemical group having an amino group attached to an alkyl group. Preferably an amino alkyl is of the structure —(CH₂)ₙNH—, wherein n is an integer ranging from about 2 to about 10. More preferably it is of the structure —(CH₂)ₙNH—, wherein n is an integer ranging from about 2 to about 4. Most preferably, the amino alkyl group is of the structure —(CH₂)₂NH—.

“Nucleic acid” refers to a polymer comprising 2 or more nucleotides and includes single-, double- and triple stranded polymers. “Nucleotide” refers to both naturally occurring and non-naturally occurring compounds and comprises a heterocyclic base, a sugar, and a linking group, preferably a phosphate ester. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

The phrase “coupled to a support” means bound directly or indirectly thereto including attachment by covalent binding, hydrogen bonding, ionic interaction, hydrophobic interaction, or otherwise.

“Probe” refers to a nucleic acid, such as an oligonucleotide, that can be used to detect, by hybridization, a target nucleic acid. Preferably, the probe is complementary to the target nucleic acid along the entire length of the probe, but hybridization can occur in the presence of one or more base mismatches between probe and target or in the presence of one or more universal base analogs. A probe includes a surface-immobilized molecule that can be recognized by a particular target. See U.S. Pat. No. 6,582,908, incorporated herein by reference in its entirety for all purposes, for an example of arrays having all possible combinations of probes with 10, 12 or more bases.

“Perfect match probe” refers to a probe that has a sequence that is perfectly complementary to a particular target sequence. The test probe is typically perfectly complementary to a portion (subsequence) of the target sequence. The perfect match (PM) probe can be a “test probe,” a “quality control probe,” a “normalization control” probe, an expression level control probe and the like. A perfect match control or perfect match probe is, however, distinguished from a “mismatch control” or “mismatch probe.” In the case of expression monitoring arrays, perfect match probes are typically preselected (designed) to be complementary to particular sequences or subsequences of target nucleic acids (e.g., particular genes). In contrast, in generic difference screening arrays, the particular target sequences are typically unknown. In the latter case, perfect match probes cannot be preselected. The term perfect match probe in this context is to distinguish that probe from a corresponding “mismatch control” that differs from the perfect match in one or more particular preselected nucleotides as described below.

“Mismatch control” or “mismatch probe,” in expression monitoring arrays, refers to probes whose sequence is deliberately selected not to be perfectly complementary to a particular target sequence. For each mismatch (MM) control in a high-density array there preferably exists a corresponding perfect match (PM) probe that is perfectly complementary to the same particular target sequence. In “generic” (e.g., random, arbitrary, haphazard, etc.) arrays, since the target nucleic acid(s) are unknown perfect match and mismatch probes cannot be a priori determined, designed, or selected. In this instance, the probes are preferably provided as pairs where each pair of probes differ in one or more preselected nucleotides. Thus, while it is not known a priori which of the probes in the pair is the perfect match, it is known that when one probe specifically hybridizes to a particular target sequence, the other probe of the pair will act as a mismatch control for that target sequence. It will be appreciated that the perfect match and mismatch probes need not be provided as pairs, but may be provided as larger collections (e.g, 3, 4, 5, or more) of probes that differ from each other in particular preselected nucleotides. While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions. In a particularly preferred embodiment, perfect matches differ from mismatch controls in a single centrally-located nucleotide.

“Labeled moiety” refers to a moiety capable of being detected by the various methods discussed herein or known in the art.

The term “oligonucleotide,” sometimes referred to as “polynucleotide,” includes, but is not limited to, a nucleic acid ranging from at least 5, 10, 20 or 25 bases long and may be up to 20, 50, 100, 1,000, or 5,000 bases long and/or a compound that specifically hybridizes to a polynucleotide. A polymorphic site can occur within any position of the oligonucleotide. Oligonucleotides of the present invention include sequences of deoxyribonucleic acid (DNA) or ribonucleic acid (RNA) and mimetics thereof which may be isolated from natural sources, recombinantly produced or artificially synthesized. A further example of a polynucleotide of the present invention may be peptide nucleic acid (PNA). See U.S. Pat. No. 6,156,501, incorporated herein by reference in its entirety for all purposes. The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The terms “solid support,” “support,” and “substrate” as used herein are used interchangeably and include, but are not limited to, a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305, incorporated herein by reference in its entirety for all purposes, for exemplary substrates.

In a certain embodiment, the array of oligonucleotide probes is a high density array comprising greater than about 100, greater than about 1,000, greater than about 16,000, or greater than about 65,000 or 250,000 or even 1,000,000 different oligonucleotide probes. Such high density arrays comprise a probe density of generally greater than about 60, more generally greater than about 100, most generally greater than about 600, often greater than about 1000, more often greater than about 5,000, most often greater than about 10,000, greater than about 40,000, greater than about 100,000, or greater than about 400,000 different oligonucleotide probes per cm². The oligonucleotide probes range from about 5 to about 50 nucleotides, from about 10 to about 40 nucleotides, or from about 15 to about 40 nucleotides in length. Although a planar array surface is typically used, the array may be fabricated on a surface of virtually any shape or even a multiplicity of surfaces. High density arrays of the invention are further described in U.S. Pat. No. 6,040,138, incorporated herein by reference in its entirety for all purposes.

In certain embodiments, the present invention employs the use of genomic samples. As used herein, the term “genomic sample” is intended to include, but is not limited to, a sample of genomic nucleic acids such as genomic DNA. “Genomic DNA” is intended to include, but is not limited to, DNA such as nuclear DNA (i.e., DNA in the chromosomes of an organism, e.g., genetic material corresponding to one chromosome, two or more chromosomes or all chromosomes present in an organism, sample or cell), mitochondrial DNA, sperm cell DNA, egg cell DNA and the like. As used herein, the term “whole genomic DNA” is intended to include, but is not limited to, genetic material corresponding to all chromosomal DNA sequences in an organism, sample or cell or all mitochondrial DNA sequences in an organism, sample or cell or both all chromosomal DNA and all mitochondrial DNA sequences in an organism, sample or cell.

A genomic sample may be obtained from a variety of sources including a biological fluid sample (e.g., serum, sputum, urine), biological tissue sample (e.g., a biopsy) or biological cell sample (e.g., a cheek scraping). As used herein, the term “biological sample” is intended to include, but is not limited to: tissues, cells and biological fluids isolated from a subject; tissues, cells and fluids present within a subject; as well as tissues, cells and biological fluids isolated from a subject and maintained in culture. Biological samples may be of any biological tissue or fluid or cells. Typical biological samples include, but are not limited to, sputum, lymph, blood, blood cells (e.g., white cells), fat cells, cervical cells, cheek cells, throat cells, mammary cells, muscle cells, skin cells, liver cells, spinal cells, bone marrow cells, tissue (e.g., muscle tissue, cervical tissue, skin tissue, spinal tissue, liver tissue and the like) fine needle biopsy samples, urine, cerebrospinal fluid, peritoneal fluid and pleural fluid, or cells therefrom. Biological samples may also include sections of tissues such as frozen sections taken for histological purposes or formalin-fixed, paraffin-embedded tissue. A biological sample may be obtained from a mammal, including, but not limited to horses, cows, sheep, pigs, goats, rabbits, guinea pigs, rats, mice, gerbils, non-human primates and humans. Biological samples may also include cells from microorganisms (e.g., bacterial cells, viral cells, yeast cells and the like) and portions tLereol. As used herein, the term “biological fluid” is intended to include any fluid taken from a biological organism. Biological fluids include, but are not limited to, sputum, lymph, blood, urine, tears, breast milk, nipple aspirate fluid, seminal fluid, vaginal secretions, cerebrospinal fluid, peritoneal fluid, pleural fluid, pus, ascites and the like.

A genomic sample (e.g., genomic DNA) may be extracted and purified using a variety of methods known in the art. For example, a variety of commercial kits and protocols are available to isolate genomic DNA: DYNABEADS® (Dynal Biotech, Carlsbad, Calif.); Phase Lock Gel procedure (Eppendorf, Westbury, N.Y.); SV Total RNA Isolation System (Promega, Madison, Wis.); DNeasy® Tissue Kit (Qiagen, Valencia, Calif.); QIAzol™ Lysis Reagent (Qiagen, Valencia, Calif.); and Mitochondrial Isolation Kit (Sigma-Aldrich, St.

Louis, Mo.), each of which is incorporated herein by reference in its entirety for all purposes.

Primers may be designed using standard techniques. For example, a computer program is available on the internet at the Operon Technologies, Inc. website. The Operon Oligo Toolkit allows a user to input a potential primer sequence into the webform. The site will instantly calculate a variety of attributes for the oligonucleotide including molecular weight, GC content, Tₘ, and primer-dimer sets. One of skill in the art may also plot the oligonucleotide against a second sequence.

A number of methods disclosed herein require fragmenting and/or labeling the nucleic acid sample, e.g., genomic DNA. Methods for fragmentation and labeling nucleic acids for hybridization to nucleic acid arrays are described in U.S. Patent Publication No. 20050191682, incorporated herein by reference in its entirety for all purposes. In certain aspects, the fragmentation method used is an alternative to methods that use DNase I, such as those described in Wodicka et al. (1997) Nature Biotech. 15:1359 and Matsuzaki et al. (2004) Gen. Res. 14:414, each of which is incorporated herein by reference in its entirety for all purposes. In many aspects, DNA is amplified to generate an amplified DNA sample and the amplified sample is subjected to random fragmentation and labeling of fragments with a detectable label, such as biotin. The labeled fragments are hybridized to an array and the hybridization pattern may be detected and analyzed to obtain information about the starting sample. In certain aspects, amplified samples are fragmented in preparation for labeling and hybridization to nucleic acid probe arrays. In one aspect, the methods include a fragmentation step and a labeling step that may occur sequentially or simultaneously. In some aspects, the fragmentation generates ends that are compatible with known methods of labeling nucleic acids, but in other aspects the fragments are subsequently treated to generate ends compatible with labeling. Certain fragmentation methods may generate a mixture of ends and the mixture may be subsequently treated to generate ends compatible with labeling. In a certain embodiment, the fragmentation and subsequent processing steps result in fragments that have a 3′ OH, and the fragments are substrates for end-labeling with terminal deoxynucleotidyl transferase (TdT).

In one aspect, fragmentation of nucleic acids comprises breaking nucleic acid molecules into smaller fragments. Fragmentation of nucleic acids may be desirable to optimize the size of nucleic acid molecules for subsequent analysis and to minimize three dimensional structure. For example, fragmented nucleic acids allow more efficient hybridization of target DNA to nucleic acid probes than non-fragmented DNA and fragmented DNA that is to be end labeled allows for the incorporation of additional labels. According to a certain embodiment, before hybridization to a microarray, target nucleic acid is fragmented to sizes ranging from about 40 to about 200 bases long, or from about 50 to about 150 bases long, to improve target specificity and sensitivity. In some aspects, the average size of fragments obtained is at least 10, 20, 30, 40, 50, 60, 70, 80, 100 or 200 bases and less than 300 bases. If the fragments are double stranded, this length refers to base pairs and if single stranded this length refers to bases. Conditions of the fragmentation reaction may be optimized to select for fragments of a desired size range. One of skill in the art will recognize that a nucleic acid sample when fragmented will result in a distribution of fragment sizes, preferably the distribution is centered about a selected length, for example, the center of the distribution of fragment sizes may be about 20, 40, 50, 60, 70, 80 or 100 bases or base pairs. In a certain aspect the methods reproducibly generate fragments that have approximately the same size distribution.

Chemical fragmentation methods that may be used include, for example, hydrolysis catalyzed by metal ion complexes, such as Cu²⁺ and Ce²⁺ complexes; oxidative cleavage by metal ion complexes, such as Fe²⁺ and Cu²⁺ complexes, photochemical cleavage, and acid-catalzyed depurination followed by AP endonuclease, heat or base treatment. Fragments may be labeled enzymatically or chemically. Chemical DNA labeling methods that may be used include incubation with a reactive reagent, such as, biotin-amine, biotin-hydrazides, diazo-biotin, biotin-platinum, biotin-psoralen, and biotin-aryl azide methods.

In some aspects, hydrolysis methods generate 5′ phosphates and 3′ hydroxyl ends which are compatible with labeling methods such as end labeling with terminal transferases and oxidative methods generate 5′ and 3′ carbonyl residues. Carbonyls may be chemically labeled, for example, with biotin-amines and -hydrazides. The phosphate backbone may be labeled, for example, with diazo-biotin and specific bases can be labeled, for example, with biotin-piatinum, -psoralen and -aryl azide.

In another aspect the fragments are an amplification product resulting from a whole genome sampling assay (WGSA) which is described, for example, in U.S. Patent Publication Nos. 20040146890 and 20040067493, incorporated herein by reference in their entirety for all purposes. In general, genomic DNA is fragmented with one or more restriction enzymes, adaptors are ligated to the fragments and the adaptor ligated fragments are subjected to PCR amplification using a primer to the adaptor sequence. The PCR preferentially amplifies fragments that are less than about 2 kb and greater than about 200 base pairs so a representative subset of the genome is amplified. The disclosed chemical fragmentation methods may be used to fragment the resulting WGSA amplification product prior to end labeling and hybridization to an array, for example, a genotyping array.

In general, a restriction enzyme recognizes a specific nucleotide sequence of four to eight nucleotides (though this number can vary) and cuts a DNA molecule at a specific site. For example, the restriction enzyme EcoRI recognizes the sequence GAATTC and will cut a DNA molecule between the G and the first A. Many different restriction enzymes are known and appropriate restriction enzymes can be chosen for a desired result. For a thorough explanation of the use of restriction enzymes, see for example, section 5, specifically pages 5.2-5.32 of Sambrook et al., supra, incorporated herein by reference in its entirety for all purposes. In certain aspects of the invention, the restriction endonucleases NspI or Styl will be utilized.

After digestion, adaptor sequences can be ligated to the fragments. Adaptor sequences are generally oligonucleotides of at least 5 or 10 bases and usually no more than 50 or 60 bases in length, however, adaptor sequences may be even longer, up to 100 or 200 bases depending upon the desired result. For example, if the desired outcome is to prevent amplification of a particular fragment, longer adaptor sequences designed to form stem loops or other tertiary structures may be ligated to the fragment. Adaptor sequences may be synthesized using any methods known to those of skill in the art. For the purposes of this invention one may comprise templates for PCR primers and/or tag or recognition sequences. The design and use of tag sequences is described in U.S. Pat. No. 5,800,992 and U.S. Provisional Patent Application No. 60/140,359, filed Jun. 23, 1999, both of which are incorporated by reference for all purposes. Adaptor sequences may be ligated to either blunt end or sticky end DNA. Methods of ligation will be known to those of skill in the art and are described, for example, in Sambrook et al., supra, incorporated herein by reference in its entirety for all purposes. Methods include DNase digestion to “nick” the DNA, ligation with ddNTP and the use of polymerase I to fill in gaps or any other methods described in the art.

Examples of chemical methods useful in the fragmentation of DNA according to the disclosed methods include: hydrolytic methods (see, for example, Sreedhara et al. (2000) J. Amer. Chem. Soc. 122:8814); oxidative-based metallo-nucleases (see, for example, Pogozelski and Tullius (1998) Chem. Rev. 98:1089 and James G. Muller et al., Chem. Rev. 1998, 98:1109-1151); photocleavage (see, for example, Nielson (1992) Amer. Chem. Soc. 114:4967); acid catalyzed depurination, (see, for example, Proudnikov and Mirzabekov, Nucleic Acids Res. 1996, 24, 4535-4532); alkylation (see, for example, Kenneth A. Browne, Amer. Chem. Soc. 2002, 124, 7950-7962); and fragmentation facilitated by reagents used in Maxam-Gilbert type sequencing methods. Fragmentation of DNA in low salt buffers at pH 6-9 has also been reported, see, for example, WO 03/050242 A2, US 20030143599 and US 20040209299, each of which is incorporated herein by reference in its entirety for all purposes.

In a certain embodiment, the hybridized nucleic acids are detected via one or more labels attached to the sample nucleic acids. In a certain aspect the fragments are end labeled using a terminal transferase enzyme (e.g., TdT). TdT catalyzes the template independent addition of deoxy- and dideoxynucleoside triphosphates to the 3′ OH ends of double- and single-stranded DNA fragments and oligonucleotides. TdT can also add homopolymers of ribonucleotides to the 3′ end of DNA. The preferred substrate for TdT is a protruding 3′ end but the enzyme will also add nucleotides to blunt and 3′-recessed ends of DNA fragments. The enzyme uses cobalt as a cofactor.

Terminal transferase may be used to incorporate, for example, digoxigenin-, biotin-, and fluorochrome-labeled deoxy- and dideoxynucleoside triphosphates as well as radioactive labeled deoxy- and dideoxynucleoside triphosphates. In a certain embodiment, a biotinylated compound is added by TdT to the 3′ end of the DNA. In a certain aspect fragments are labeled with biotinylated compounds such as those disclosed in U.S. patent Publication No. 20030180757, incorporated herein by reference in its entirety for all purposes. The biotin may be detected by contacting it with streptavidin with a fluorescent conjugate, such as streptavidin-phycocrythrin (Molecular Probes, Eugene, OR). A number of labeled and unlabeled streptavidin conjugates are available. Conjugates include fluorescent dyes such as fluorescein and rhodamine and phycobiliproteins such as phycoerythrin. Biotinylated antibodies to streptavidin may be used to amplify signal. For additional labeling methods and compounds see, for example, U.S. Pat. Nos. 4,520,110 and 5,055,556 and U.S. Patent Pub. No. 20040002595, each of which is incorporated herein by reference in its entirety for all purposes.

In certain aspects, the 3′ end of fragments that are modified, for example, with a phosphoglycolate or 2′ deoxyribolactone, may be labeled using a 3′ end repair system, tailing with dGTP/GTP and labeling with a DNA labeling reagent using TdT. This is described in WO 03/050242, incorporated herein by reference in its entirety for all purposes. In certain aspects, fragments may be labeled by disproportionation and exchange of a labeled nucleotide to the 3′ end by TdT in the presence of metal ions Co²+, Mn²⁺ or Mg²⁺, Co²⁺ being preferred, as described in Anderson et al. (1999) Nucleic Acids Res. 27:3190, incorporated herein by reference in its entirety for all purposes. Optimal concentration of the metal ion is 1-2 mM.

In another embodiment, the label is simultaneously incorporated during an amplification step in the preparation of the sample nucleic acids. Thus, for example, PCR may be performed with labeled primers or labeled nucleotides to provide a labeled amplification product. Alternatively, a label may be added directly to the original nucleic acid sample or to the amplification product after the amplification is completed. Means of attaching labels to nucleic acids are well known to those of skill in the art.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like), luminescent and bioluminescent markers (e.g., biotin, luciferase (e.g., bacterial, firefly, click beetle and the like), luciferin, aequorin and the like), radiolabels (e.g., ³H, ¹²⁵I, ³⁵S, ¹⁴C, or ³²P), enzymes (e.g., galactosidases, glucorinidases, phosphatases (e.g., alkaline phosphatase), peroxidases (e.g., horseradish peroxidase), cholinesterases and the like), and calorimetric labels such as colloidal gold or colored glass or plastic (e.g., polystyrene, polypropylene, latex, and the like) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference in its entirety for all purposes.

Means of detecting such labels are well known to those of skill in the art. Thus, for example, radiolabels may be detected using photographic film or scintillation counters, fluorescent markers may be detected using a photo detector to detect emitted light. Enzymatic labels are typically detected by providing the enzyme with a substrate and detecting the reaction product produced by the action of the enzyme on the substrate, and calorimetric labels are detected by simply visualizing the colored label.

The label may be added to the target (sample) nucleic acid(s) prior to, or after the hybridization. “Direct labels,” as used herein, are detectable labels that are directly attached to or incorporated into the target (sample) nucleic acid prior to hybridization. In contrast, “indirect labels,” as used herein, are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind tne biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: “Hybridization With Nucleic Acid Probes,” P. Tijssen, ed. Elsevier, N.Y., (1993), incorporated herein by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170, each of which is incorporated herein by reference in its entirety for all purposes.

Additionally, the present invention may have preferred embodiments that include methods for providing genetic information over networks such as the internet as shown in U.S. Ser. Nos. 10/197,621, 10/063,559 (United States Publication No. 20020183936), Ser Nos. 10/065,856, 10/065,868, 10/328,818, 10/328,872, 10/423,403, and 60/482,389, each of which is incorporated herein by reference in its entirety for all purposes.

The contents of all references, patents and published patent applications cited throughout this application are hereby incorporated by reference in their entirety for all purposes.

What is claimed is:

1. A method of monitoring hybridization of control sample sequences to control probe sequences on an array comprising:
providing a high density polymer probe array having a plurality of test probes that bind to test sample targets and a plurality of control probes at predefined regions on the array and wherein the control probes bind to control sample targets; the control probes further comprising a plurality of first control probes at predefined regions on the array and a plurality of second control probes at predefined regions on the array; and wherein the first control probes are different from the second control probes;
contacting first control sample targets with the array; and
determining whether hybridization occurred between the first control sample targets and the first control probes.

2. The method of claim 1 wherein the first control probes are the same at given predefined region on the array.

3. The method of claim 1 wherein the first control probes bind to synthetic polymers.

4. The method of claim 3 wherein the first control probes bind to synthetic genes.

5. The method of claim 4 wherein the first control probes bind to synthetic Tag genes.

6. The method of claim 1 wherein the second control probes bind to bacterial genes.

7. The method of claim 7 wherein the bacterial genes are BioB, BioC, BioD or cre.

8. The method of claim 1 wherein the array comprises greater than 100 probes.

9. The method of claim 1 wherein the array comprises greater than 1000 probes.

10. The method of claim 1 wherein the array comprises greater than 16,000 probes.

11. The method of claim 1 wherein the array comprises greater than 65,000 probes.

12. The method of claim 1 wherein the array comprises greater than 250,000 probes.

13. The method of claim 1 wherein the array comprises greater than 1,000,000 probes.

14. The method of claim 1 wherein the array comprises greater than 100 probes per cm².

15. The method of claim 1 wherein the array comprises greater than 1000 probes per cm².

16. The method of claim 1 wherein the array comprises greater than 10,000 probes per cm².

17. The method of claim 1 wherein the array comprises greater than 40,000 probes per cm².

18. The method of claim 1 wherein the array comprises greater than 100,000 probes per cm².

19. The method of claim 1 wherein the array comprises greater than 400,000 probes per cm².

20. The method of claim 1 wherein the test probes are oligonucleotides.

21. A method for quality control of an oligonucleotide array having probes thereon, wherein said array has a plurality of quality control blocks surrounded at the four corners of each with first checkerboard features, said method comprising,
providing a quality control block in the center of the array;
placing at the four corners of the center control block, second checkerboard features, wherein control sample sequences that bind to the second checkerboard features do not bind to the first checkerboard features;
imaging the center control blocks and the second checkerboard features to allow for gridding of the array and a determination of whether the probes in the quality control blocks have been accurately synthesized.

22. The method of claim 21 wherein the second checkerboard features are the same at a given predefined region on the array.

23. The method of claim 21 wherein the second checkerboard features bind to synthetic polymers.

24. The method of claim 21 wherein the second checkerboard features bind to synthetic genes.

25. The method of claim 21 wherein the second checkerboard features bind to synthetic Tag genes.

26. The method of claim 21 wherein the array comprises greater than 100 probes.

27. The method of claim 21 wherein the array comprises greater than 1000 probes.

28. The method of claim 21 wherein the array comprises greater than 16,000 probes.

29. The method of claim 21 wherein the array comprises greater than 65,000 probes.

30. The method of claim 21 wherein the array comprises greater than 250,000 probes.

31. The method of claim 21 wherein the array comprises greater than 1,000,000 probes.

32. The method of claim 21 wherein the array comprises greater than 100 probes per cm².

33. The method of claim 21 wherein the array comprises greater than 1000 probes per cm².

34. The method of claim 21 wherein the array comprises greater than 10,000 probes per cm².

35. The method of claim 21 wherein the array comprises greater than 40,000 probes per cm².

36. The method of claim 21 wherein the array comprises greater than 100,000 probes per cm².

37. The method of claim 21 wherein the array comprises greater than 400,000 probes per cm².

38. The method of claim 21 wherein the probes are oligonucleotides.
